(19) United States
(12) Patent Application Publication
(10) Pub. No.: US 2025/0116635 A1
(43) Pub. Date: Apr. 10, 2025

(54) APPARATUS FOR FAST GAS CHROMATOGRAPHY AND INFRARED SPECTROSCOPY MEASUREMENTS OF OILFIELD FLUIDS

(71) Applicant: Schlumberger Technology Corporation, Sugar Land, TX (US)

(72) Inventors: Mahdi Ammar, Clamart (FR); Dariusz Strapoc, Le Plessis-Robinson (FR)

(21) Appl. No.: 18/906,415

(22) Filed: Oct. 4, 2024

(30) Foreign Application Priority Data
Oct. 4, 2023 (EP) 23306705.7

(51) Int. Cl. G01N 30/02; G01N 30/74; G01N 33/22

(52) U.S. Cl. CPC G01N 30/02; G01N 30/74; G01N 33/225; G01N 2030/025

(57) ABSTRACT

An apparatus for making fast gas chromatography measurements of an oilfield gas includes an infrared laser configured to emit an infrared laser beam; an infrared sensor configured to receive the infrared laser beam; a Fabry-Perot gas cell deployed in a path between the infrared laser and the infrared sensor such that the infrared laser beam passes through the gas cell, the gas cell configured to receive the gas sample; a gas chromatography column assembly including an input port, a gas chromatography column, and an output port, the gas chromatography column assembly configured to provide the gas sample to the Fabry-Perot gas cell; and a controller in electronic communication with the infrared sensor and configured to process the received infrared laser beam to estimate a composition of the gas sample.

### CROSS REFERENCE TO RELATED APPLICATIONS

This application claims priority to and the benefit of European Patent Application No. 23306705.7, titled “Apparatus for Fast Gas Chromatography and Infrared Spectroscopy Measurements of Oilfield Fluids,” filed Oct. 4, 2023, the entire disclosure of which is hereby incorporated herein by reference.

### BACKGROUND

Gas chromatography (GC) is a widely used technique for separating and analyzing chemical compounds that can be vaporized, such as organic compounds. Common applications relate to the quantitative and/or qualitative analysis of food composition, natural products, food additives, flavor and aroma components such as essential oils, a variety of transformation products and contaminants, such as pesticides, fumigants, natural toxins, pharmaceuticals, packaging materials, and environmental pollutants. GC techniques are also used to evaluate the composition of gases that are liberated during downhole drilling operations, for example, including methane (C1), ethane (C2), propane (C3), butane (C4), pentane (C5) and the like, as well as alkenes and other compounds.

Such measurements may provide valuable information to a mud logger and may provide information about the maturity and nature of hydrocarbons in the reservoir, compartmentalization of intervals in the reservoir being drilled, and oil quality, as well as information regarding production zones, lithology changes, history of reservoir accumulation, seal effectiveness, and environmental impact of the drilling operation.

In mud logging, there is a need in the industry for improving the speed and sensitivity of gas composition measurements, for example, for making rapid measurement of the composition of natural gas as it is brought to the surface during oil or gas drilling. Improving the speed and sensitivity of such measurements may help geologists and drilling engineers to more quickly identify the presence of natural gas deposits and to monitor the composition of the gas during drilling operations and to therefore make more timely drilling decisions.

### BRIEF DESCRIPTION OF THE DRAWINGS

For a more complete understanding of the disclosed subject matter, and advantages thereof, reference is now made to the following descriptions taken in conjunction with the accompanying drawings, in which:

FIG. 1 depicts an example drilling rig including a fast gas chromatography apparatus for evaluating formation gas composition.

FIG. 2 depicts an example embodiment of the fast GC apparatus shown on FIG. 1.

FIG. 3 depicts one example of a macro Fabry-Perot cell suitable for use in the fast GC apparatus shown on FIGS. 1 and 2.

FIGS. 4A and 4B depict one example of a micro Fabry-Perot cell suitable for use in the fast GC apparatus shown on FIGS. 1 and 2.

FIGS. 5A and 5B depict an example micro Fabry-Perot cavity in which mirrors are integrated into optical fiber ends.

FIG. 6 depicts another embodiment of a micro Fabry-Perot gas cell.

FIG. 7 depicts an alternative embodiment including multiple IR lasers, a Fabry-Perot gas cell, and an IR detector.

### DETAILED DESCRIPTION

Embodiments of this disclosure include apparatuses and methods for making fast GC and IR spectroscopy measurements and for estimating a composition of an oilfield gas sample. In one example embodiment an apparatus includes an infrared laser configured to emit an infrared laser beam; an infrared sensor configured to receive the infrared laser beam; a Fabry-Perot gas cell deployed in a path between the infrared laser and the infrared sensor such that the infrared laser beam passes through the gas cell, the gas cell configured to receive the gas sample; a gas chromatography column assembly including an input port, a gas chromatography column, and an output port, the gas chromatography column assembly configured to provide the gas sample to the Fabry-Perot gas cell; and a controller in electronic communication with the infrared sensor and configured to process the received infrared laser beam to estimate a composition of the gas sample.

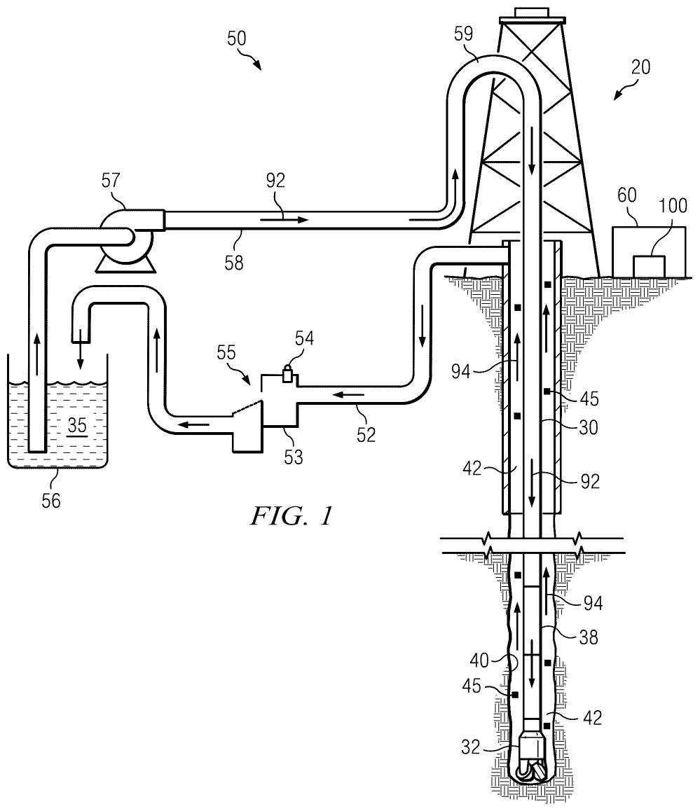

FIG. 1 depicts an example drilling rig 20 including a fast gas chromatography apparatus 100 configured to evaluate a formation gas composition. By “fast” GC apparatus it is meant that the apparatus is configured to make molecular composition and/or isotopic ratio measurements for C1 (methane) through C5 (pentane) oilfield gas compositions in less than about 1 minute. Stated another way, the disclosed fast GC apparatus may have a temporal resolution of less than about one minute. The drilling rig 20 may be positioned over a subterranean formation (not shown). The rig 20 may include, for example, a derrick and a hoisting apparatus (also not shown) for raising and lowering a drill string 30, which, as shown, extends into wellbore 40 and includes, for example, a drill bit 32 and one or more downhole measurement tools 38 (e.g., a logging while drilling tool or a measurement while drilling tool) in a bottom hole assembly (BHA) above the bit 32. Suitable drilling systems, for example, including drilling, steering, logging, and other downhole tools are well known in the art.

Drilling rig 20 further includes a surface system 50 for controlling the flow of drilling fluid used on the rig (e.g., used in drilling the wellbore 40). In the example rig depicted, drilling fluid 35 is pumped downhole (as depicted at 92), for example, via a conventional mud pump 57. The drilling fluid 35 may be pumped, for example, through a standpipe 58 and mud hose 59 in route to the drill string 30. The drilling fluid 35 typically emerges from the drill string 30 at or near the drill bit 32 and creates an upward flow 94 of mud through the wellbore annulus 42 (the annular space between the drill string and the wellbore wall). The drilling fluid 35 then flows through a return conduit 52 to a mud pit system 56 where may be recirculated. It will be appreciated that the terms drilling fluid and mud are used synonymously herein.

The circulating drilling fluid 35 is intended to perform many functions during a drilling operation, one of which is to carrying drill cuttings 45 to the surface (in upward flow 94). The drill cuttings 45 are commonly removed from the returning mud via a shale shaker 55 (or other similar solids control equipment) in the return conduit (e.g., immediately upstream of the mud pits 56). Formation gases that are released during drilling may also be carried to the surface in the circulating drilling fluid. These gasses are commonly removed from the fluid, for example, via a degasser 54 (or a gas trap) located in or near a header tank 53 that is immediately upstream of the shale shaker 55 in the example depiction. The drill cuttings 45 and gas are commonly examined at the surface to evaluate the formation layers though which the wellbore is drilled.

As is known to those of ordinary skill in the art, the formation gas may be released into the wellbore 40 via the drilling process (e.g., crushing the formation rock by the mechanical action of the drill bit) and may also migrate into the wellbore 40, for example, via fractures in the formation rock. Once in the wellbore, the formation gas may be transported to the surface via the drilling fluid (in the upwardly flowing fluid 94). The formation gas may be in solution in the drilling fluid and/or in the form of bubbles and may be sampled in the surface system, for example, via one or more drilling fluid degassers 54 and/or a head space gas probe. The disclosed embodiments are expressly not limited in regards to how the gas is sampled.

With further reference to FIG. 1, drilling rig 20 may further include a testing facility 60 (e.g., a laboratory trailer including one or more instruments suitable for making various measurements of drill cuttings and formation gases in the drilling fluid). In the depicted embodiment, the testing facility 60 includes a fast gas chromatography apparatus 100 (described in more detail below) configured to receive the formation gas and evaluate the formation gas composition. The testing facility 60 may, of course, include numerous other testing instruments known to those of ordinary skill in the industry. While the following embodiments are described with respect to drilling operations, it will be understood the that the disclosed embodiments are not so limited and may be applied to the use of GC in other chemical applications.

While GC is a powerful chemical analysis technique that enables many (most) chemical species to be separated at the detector, there remains room for improvement. For example, conventional GC measurements can be time consuming or not may not be well suited to making multiple successive measurements of a gas stream at a fast time interval (e.g., on the order of seconds or minutes depending on the measured gases and accuracy required). The disclosed embodiments may advantageously provide for such rapid GC measurements.

It will of course be appreciated that while FIG. 1 depicts a land rig 20, that the disclosed embodiments are equally well suited for land rigs or offshore rigs. As is known to those of ordinary skill, offshore rigs commonly include a platform deployed atop a riser that extends from the sea floor to the surface. The drill string extends downward from the platform, through the riser, and into the wellbore through a blowout preventer (BOP) located on the sea floor. The disclosed embodiments are expressly not limited in these regards.

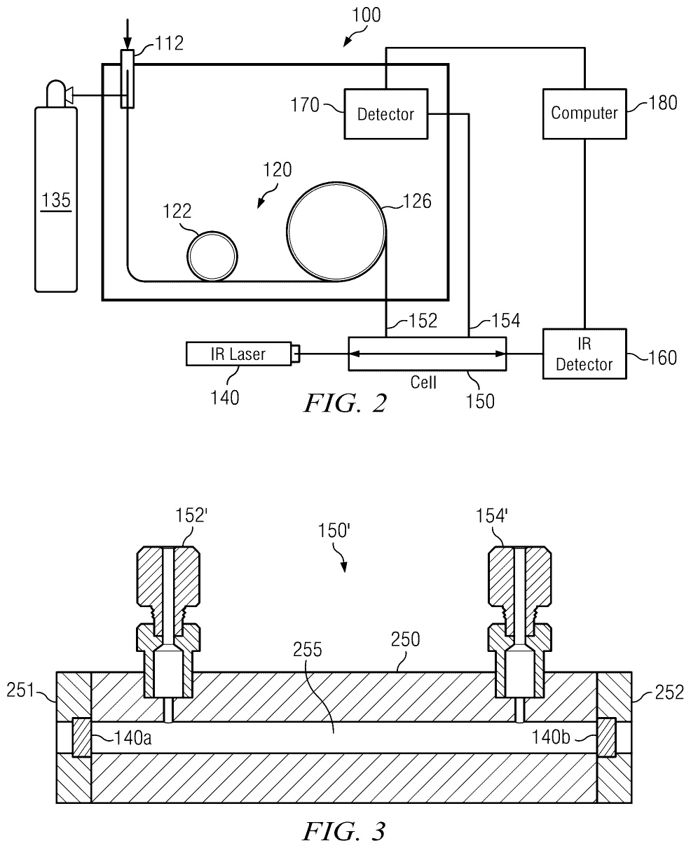

FIG. 2 depicts an example fast GC apparatus 100 including a gas sample injection port (such as an auto-injector with a sample loop) 112 configured to feed a gas sample into a column assembly 120 including an optional precut column 122 and a main GC column 126. The GC apparatus 100 further includes a carrier gas supply 135, such as a supply of compressed nitrogen, argon, helium, or air. An injected (e.g., auto-injected) gas sample is mixed with the carrier gas and transported through the column assembly 120. The optional precut column 122 may be configured to remove heavier hydrocarbon compounds having a number of carbon atoms above a threshold, such as C6, C8, or C10 and above. The main column 126 generally includes a stationary phase and is intended to separate the various gas compounds in the gas sample such that they exit (or depart) the main column 126 at distinct elution times, for example, such that C1 exits the column before C2, which exits the column before C3, and so on.

The apparatus 100 further includes a Fabry-Perot (FP) gas cell 150 configured to receive the output gas from the main column 126. Example embodiments of the Fabry-Perot gas cell 150 may include inlet and outlet ports 152 and 154 configured to receive and release the stream of gas into and out of the cell 150. An infrared laser 140 emits infrared radiation (e.g., an infrared laser beam) that passes through the FP gas cell 150 (and the gas therein) to an infrared sensor 160 configured to receive the infrared laser beam. The sensor 160 is in electronic communication with a controller 180 that is configured to process the detected infrared radiation and generate an IR spectrum. The controller 180 may be further configured to estimate a molecular composition of the gas or an elemental isotopic ratio (e.g., of carbon or hydrogen).

While not depicted on FIG. 2, it will be understood that the apparatus 100 may further include a pressure controller and a vacuum pump that may be configured to regulate the gas pressure in the Fabry-Perot gas cell 150 or that is transferred into or through the cell. In some example applications (e.g., when making isotopic measurements such as when measuring a ratio of carbon-13 methane to carbon-12 methane or carbon-13 carbon dioxide to carbon-12 carbon dioxide), it may be desirable to make the measurements at low pressures, for example, at pressures less than about 100 mbar (or even at pressures as low as about 10 mbar).

With continued reference to FIG. 2, the outlet port 154 in the Fabry-Perot gas cell 150 may optionally be in fluid communication with a GC detector 170. The GC detector may include substantially any suitable detector, for example, including a flame ionization detector (FD), a flame photometric detector (FPD), a thermal conductivity detector (TCD), or a mass spectrometer. The GC detector 170 is also in electronic communication with the controller 180, which may be further configured to estimate a composition of the gas based on the detector output. It will, of course, be appreciated that the controller 180 may include computer hardware and software configured to cause the fast GC apparatus 100 to control the function of the apparatus. The hardware may include one or more processors (e.g., microprocessors) which may be connected to one or more data storage devices (e.g., hard drives or solid state memory) and user interfaces. It will be further understood that the disclosed embodiments may include processor executable instructions stored in the data storage device. The disclosed embodiments are, of course, not limited to the use of or the configuration of any particular computer hardware and/or software.

The fast GC apparatus 100 may include substantially any suitable IR source, for example, including an infrared laser 140 that emits in the near-IR or mid-IR wavelengths (e.g., in the 1-2 m wavelength range). Suitable lasers may include, for example, a tunable telecom grade laser.

As noted above the disclosed embodiments are directed to an apparatus for making fast GC and IR spectroscopy measurements. One aspect of the disclosed embodiments was the realization that integrating a low-volume Fabry-Perot cell with a high efficiency column and sensitive detectors may enable fast GC and IR spectroscopy measurements.

With continued reference to FIG. 2, apparatus 100 may advantageously make use of fast, low-volume injection techniques. For example, the injection port 112 may include an auto-injector with a low-volume sample loop (e.g., less than 5 ml, less than 1 ml, less than 100 μL, or even less than 10 μL). In example embodiments, the injection port 112 may be a splitless or on-column injection port to reduce the time required for sample introduction and increase the speed of the analysis.

The fast GC apparatus 100 may further advantageously include a main column 126 that has a short length and/or a small inner diameter such that fast separation times can be achieved. The main column 126 may further include a capillary column including thin films of a stationary phase coating the inner surface of the column. The use of such a stationary phase may further improve the separation efficiency of the column and reduce the time required to achieve suitable separation.

The apparatus may further include a heating or cooling module (not shown in FIG. 2) to heat or cool the gas sample prior to passing through the column assembly 120. The module may be configured to rapidly heat or cool the gas sample to further reduce the time required for the separation and increase the speed of the analysis. Moreover, the apparatus may make use of high carrier gas flow rates to reduce the time required for the gas to traverse the main column 126. The column efficiency may be further improved by implementing vacuum separation, for example, by operating the column 126 at a reduced outlet pressure. The apparatus 100 may therefore further include a vacuum pump configured to draw the gas sample through the column assembly 120. Such vacuum separation may be advantageously implemented with a relatively short and wide-bore column.

With still further reference to FIG. 2, the GC detector 170 may include substantially any detector that is suitable for high speed GC measurements. For example, the detector may include a mass flow detector that is responsive to the mass of sample component reaching the detector per unit time (e.g., ng/s) or a concentration-sensitive detector that is responsive to the concentration of a sample component in the mobile phase (e.g., ng/mL). Example detectors that may be well-suited fast (high-speed) GC measurements include flame ionization detectors (FID), thermal conductivity detectors (TCD), and electron capture detectors (ECD), as they have fast response times and can accommodate high flow rates. Mass spectrometers are also frequently used in high-speed GC due to their high sensitivity and selectivity, but they tend to have slower response times compared to the other detectors.

Open cell detectors and flame-based detectors, such as FID, commonly have response times of a few milliseconds or less if the capillary separation column is passed through the burner tip and positioned just below the base of the flame. For closed cell or concentration detectors, including a photoionization detector, an ECD, and a TCD, extra-column band broadening can be excessive unless specially designed devices with small cell volume are used or the detector is operated at sub-ambient pressure. At reduced column outlet pressure, the carrier gas velocity in the detector is increased, and the cell is swept out more quickly. Extra gas, called makeup gas, can be introduced into the detector cell to sweep the cell more rapidly and reduce peak broadening and distortion.

One difficulty with making fast GC measurements is achieving suitable accuracy and sensitivity for the small sample volumes required to achieve a high measurement frequencies (i.e., a fast measurement). It will be appreciated that the limit of detection (LOD) or minimum detectable level (MDL) refers to the quantity or concentration of solute, which generates a peak height (or peak area) corresponding to a signal to noise ratio (SNR) of 2 (although more recently an SNR of 3 is commonly used). These measures (LOD and MDL) are more useful than a simple response/mass value because they include information about the system noise. Moreover, MDL is peak-width independent, but the data rate or filter bandwidth should be specified.

Mass flow detectors respond to the mass of a compound in the detector at a given time and the MDL units are mass per unit time. Concentration detectors respond to the concentration of the compound in the detector sensing volume (not the sample) and their MDL units are mass/volume. For mass flow detectors, MDL is calculated by measuring the noise and the area of a peak of a known injected mass. For example, for a concentration detector, the MDL at S/N=2 may be expressed as follows:

$$MDL = \frac{2 * noise * amount injected}{area * flow through detector cell ( mL / s )}$$

Noise may be measured under “normal” operating conditions, with a column connected and carrier gas on. For example, noise may be measured over a period of time that is on the order of 10 times the peak width at half height (or 10 times the area/height ratio for a Gaussian peak). MDL may be specified at a data rate of 5 Hz, which is appropriate for peaks that are between 1.5 and 3 seconds wide at half height. Such peaks generally have from 15 to 30 data points across them from baseline to baseline.

A detector MDL may be related to the minimum detectable sample concentration (Cᵢ) for a given chromatographic method. For a mass flow detector, the minimum detectable sample concentration may be estimated as follows:

$${C}_{i} = \frac{{ω}_{i} * MDL}{{v}_{e}}$$

where ωᵢ represents the peak width measured at half height for a Gaussian peak and vₑ is the volume injected corrected by the GC injector. One example calculation follows for a C1 peak from standard mud logging that is 0.2 seconds wide at half height on the FID. Note that in this example, the measured gas is methane (C1), which has 0.75 grams of carbon for every gram of methane (CH₄). When the FID MDL is 2 pgC/s (Acq. Rate: 100 Hz), then the MDL for C1 is (2/0.75) pg/s of C1 or 2.66 pg/s. Using these values and assuming a 15 μL auto-injection (no split), yields the following (methane molecular weight (MW): 16.04 g/mol):

$${C}_{i} = \frac{0.2 * 2.66}{15} = 0.035 \frac{ng}{ml}$$

$${C}_{i} ( ppm ( v / v ) ) = {C}_{i} ( \frac{ng}{ml} ) * \frac{24.46}{MW} = 0.053 ppm ( \frac{v}{v} )$$

It will be appreciated that the above concentration calculation may further include a flow term to consider the total flow through the detector and further includes the flow through the column.

It will be further appreciated that detection limits may vary for the micro and macro Fabry-Perot gas cells. In particular, owing to the small cavity volume, the cavity length of a micro Fabry-Perot gas cell tends to be relatively short such that the IR laser-gas interaction length is limited. This can result in lower sensitivity but with the trade-off of a significantly increased measurement speed. Conversely, owing to the larger cavity volume, the cavity length of a of a macro Fabry-Perot gas cell tends to be relatively long resulting in a longer IR laser-gas interaction length. This can result in greater sensitivity but with the trade-off of a reduced measurement speed. The disclosed embodiments are, of course, not limited in these regards.

Turning now to FIG. 3, one example embodiment of a macro Fabry-Perot gas cell 150′ is depicted. In the depicted embodiment, the FP gas cell 150′ includes input and output ports 152′ and 154′ coupled with a cavity body 250. The cavity body 250 includes a cavity volume 255 in fluid communication with the input and output ports 152′ and 154′. As described above, the cell 150′ may further include first and second plane mirrors 140a′ and 140b′ at opposing ends of the cavity volume.

In advantageous embodiments, the cavity body 250 may be further configured to for use with an all fiber fast GC apparatus. For example, the cavity body may be configured to receive first and second optical fibers (not shown) at opposing ends 251 and 252 thereof. The first optical fiber may be deployed between the IR laser and a first fiber coupling 261 deployed in the first end 251 of the cavity body while a second optical fiber may be deployed between a second fiber coupling 262 deployed in the second end 252 of the cavity body and the IR detector.

In certain advantageous embodiments, a macro Fabry-Perot gas cell may be sized and shaped such that it has a cavity volume in a range from about 0.1 to about 5 milliliters (e.g., from about 0.25 to about 2.5 milliliters). Moreover, the cavity length may be in a range from about 0.5 to about 10 cm. Such a macro cavity may advantageously provide for a high sensitivity (e.g., on the order of 1 part per billion (ppb) or less.

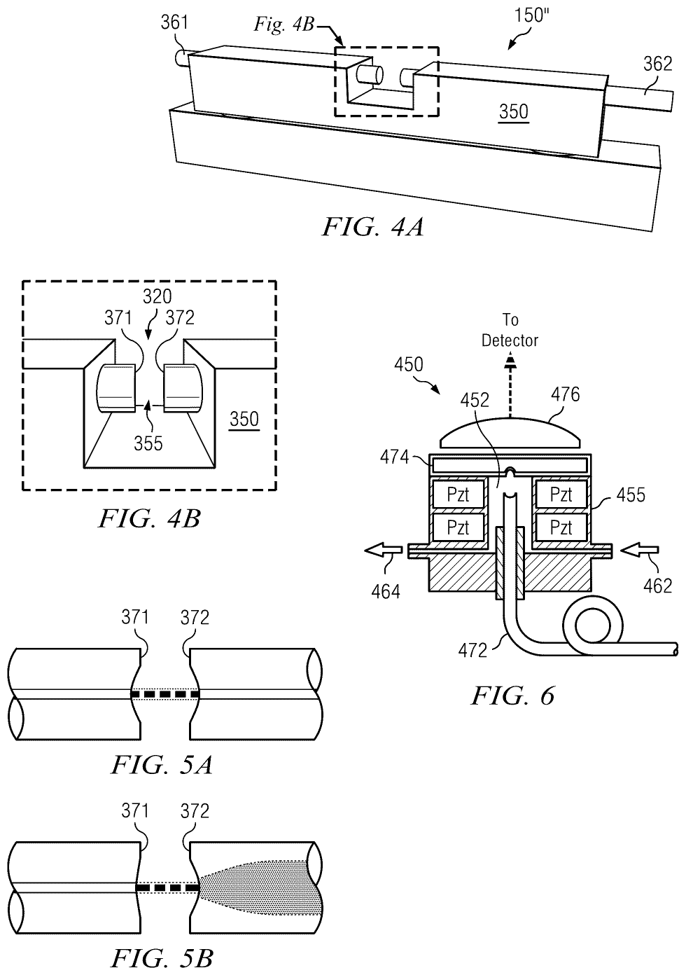

FIGS. 4A and 4B (collectively FIG. 4) depict one example embodiment of a micro Fabry-Perot gas cell 150″ suitable for use with an all fiber fast GC apparatus. In the depicted embodiment, a slot 320 is formed in a cavity body 350. First and second fibers 361 and 362 are deployed in the cavity body (e.g., embedded therein). The first optical fiber 361 may be deployed between the IR laser and the slot 320 while the second optical fiber 362 may be deployed between the slot 320 and the IR detector. The first and second fibers 361 and 362 may be positioned in the slot 320 such that a small cavity volume 355 is defined therebetween.

With continued reference to FIG. 4, mirrors may be integrated into the ends 371, 372 of the fibers 361, 362, for example, as depicted in FIGS. 5A and 5B. However, the small radii of curvature required for cavities having small mode volumes may not be within the reach of traditional polishing techniques. Successful techniques to fabricate the required small, smooth depressions (depth of order a few μm) on glass surfaces are surface etching methods and laser ablation. For example, one technique for producing curved mirror shapes with ultra-low surface roughness is CO₂-Laser ablation. The strong absorption of fused silica (SiO₂) for light between 9-11 μm wavelength is used to strongly heat the top layer of the fiber end surface by illumination with a focused CO₂ laser beam.

Subsequently to the mirror surface molding, multilayer dielectric coatings may be applied to realize low loss, high reflectivity mirrors in the visible, infrared, or ultraviolet domain (preferably infrared). For this purpose, up to tens of alternate layers of materials with high (Ta₂O₅ n=2.10) and low (SiO₂ n=1.45) index of refraction and individual thicknesses of λ/4 may be deposited on the fiber surface using ion-beam sputtering techniques. The resulting fiber-based mirrors with the shape of the original fiber surface depression can feature transmission, scattering and absorption losses down to the few parts per million, and are ready to be aligned to build high finesse, low-mode-volume resonators.

In certain advantageous embodiments, a micro Fabry-Perot gas cell may be sized and shaped such that it has a cavity volume less than about 5 microliters (e.g., less than about 1 microliter). Moreover, the cavity length may be less than about 2 mm (e.g., less than about 1 mm or less than about 0.5 mm). Such a micro cavity may advantageously provide for very rapid measurements (e.g., on the order of a few seconds or less) and good sensitivity (e.g., on the order of 1 part per million (ppm) or less).

With further reference to FIGS. 3 and 4, it will be appreciated that a Fabry-Perot cavity (or gas cell) is an optical cavity made from two parallel reflecting surfaces with some small transmissivity (e.g., thin mirrors). The Fabry Perot cavity is a type of interferometer that allows light to pass through only when it is in resonance with the cavity. To achieve appropriate resonance, the cavity length may be appropriately selected (e.g., as an integer multiple of a difference between selected peaks such as carbon-12 and carbon-13 peaks in an isotopic measurement).

Fiber Fabry-Perot Cavity (FFPC) devices may include opposing fiber mirrors, for example, as depicted on FIGS. 4, 5A, and 5B. As many applications may benefit from high Q optical resonators, the finesse is one of the key specifications for an FFPC. The reachable finesse is usually limited by the quality of the reflective coating, the surface quality, and the size of the fiber mirrors. A finesse on the order of 10′ may be advantageous. Absorption in ion beam sputtered layer systems with high reflectivity usually occurs on the order of some parts per million. Annealing of these mirrors at moderate temperatures (<350° C.) may reduce the amount of absorption even further.

Conventional FFPC realizations may pick up low frequency acoustic noise due to the large distance of the fibers from the common base. Moreover, fiber tips projecting beyond their holders into the free space introduce additional noise due to bending modes. In order to stabilize these cavity systems an electronic locking scheme with feedback bandwidths of the order of several tens of kHz may be utilized. This reduces the complexity of the assembling process and at the same time increases the passive stability. In this case, the fiber mirrors are inherently aligned by the guide provided by the cavity body. Due to the high passive stability, feedback bandwidths as low as 20 mHz may be sufficient to lock the cavity resonance to an external laser under laboratory conditions. Fast piezo tuning may enable feedback bandwidths up to 27 kHz for tight stabilization of the cavity resonance.

FIG. 6 depicts another embodiment of a micro Fabry-Perot gas cell 450. In the depicted embodiment, the cavity length and volume may be adjusted using at least one (e.g., first and second) piezoelectric transducer(s) 455. As described above with respect to FIG. 4, the micro Fabry-Perot gas cell 450 has a small cavity volume and a short cavity length. The gas sample may be received at a gas inlet port 462 and ejected at a gas outlet port 464. The micro cavity 452 may be formed between an optical fiber 472 and a mirror 474. An optional lens 476 may focus the optical output at the detector. As also described above with respect to FIGS. 3 and 4, the FP gas cell 450 may be implemented in an all fiber configuration.

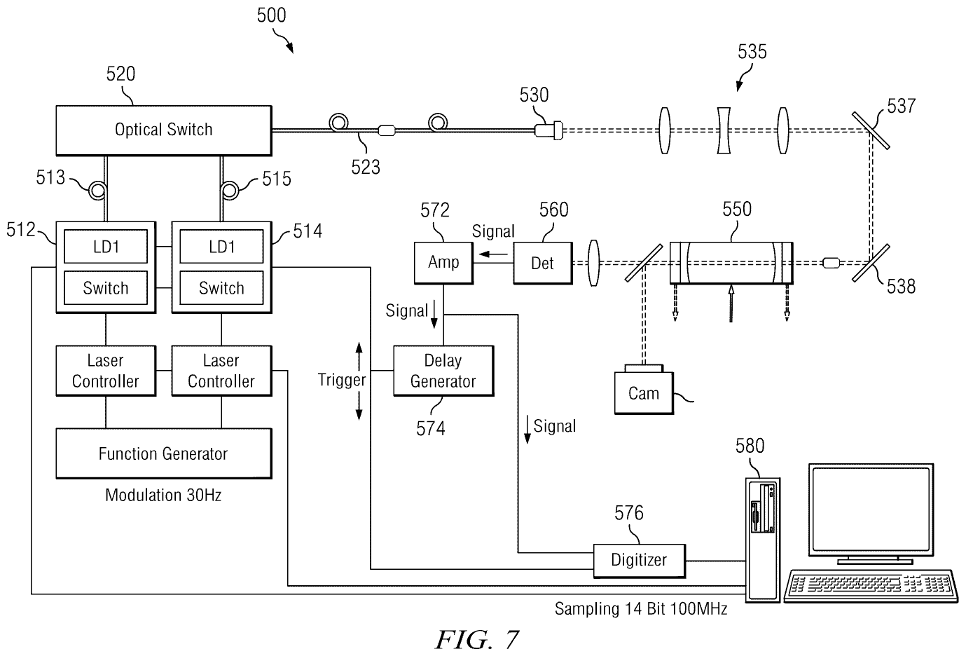

FIG. 7 depicts an alternative embodiment 500 including multiple IR lasers 512, 514, a Fabry-Perot gas cell 550, and an IR detector 560. In the depicted embodiment, first and second IR lasers 512, 514 are configured to emit IR radiation into corresponding optical fibers 513 and 515. It will be appreciated that the disclosed embodiment may include substantially any number of IR lasers, for example, one distinct IR laser for each chemical compound measured. The IR lasers 512, 514 may advantageously be tunable, so that an appropriate wavelength may be selected based upon the absorption peaks of the chemical compound. An optical switch 520 be used to select IR radiation from a desired IR laser. The switch 520 may be configured to rapidly switch between the first and second lasers 512, 514.

With continued reference to FIG. 7, the selected IR radiation continues through an optical fiber 523 to an optional collimator 530, mode matching lenses 535, mirrors 537, 538 to the Fabry-Perot gas cell 550. In an alternative all-fiber embodiment, the optical fiber 523 may be connected to the Fabry-Perot gas cell 550 (for example as described above with respect to FIGS. 3, 4, and 6). The output IR radiation may be received at the detector 560. In an all-fiber embodiment, an optical fiber may be coupled to the output and of the FP gas cell 550 and the detector 560. The embodiment 500 may further include various electronics such as an amplifier 572, a delay generator 574 that electronically couples the detector 560 and the IR lasers 512, 514, a digitizer such as an analog-to-digital converter 576, and a controller 580 such as a computer.

With still further reference to FIG. 7, it will be appreciated that embodiment 500 may further include multiple infrared detectors 560, for example, an infrared detector corresponding to each of the infrared lasers. Such an embodiment may be configured to measure multiple distinct gases simultaneously without the requirement of multiplexing the laser with an optical switch. Moreover the Fabry-Perot gas cell may be configured (e.g., the length, the finesse, and of the fiber type) for a specific gas or gases (or IR laser wavelength). Such a configuration may enable one or more micro Fabry-Perot gas cells to be integrated into a compact chip.

It will be understood that the present disclosure includes numerous embodiments. These embodiments include, but are not limited to, the following embodiments.

In a first embodiment, an apparatus for making fast gas chromatography measurements of an oilfield gas sample, the apparatus comprising an infrared laser configured to emit an infrared laser beam; an infrared sensor configured to receive the infrared laser beam; a Fabry-Perot gas cell deployed in a path between the infrared laser and the infrared sensor such that the infrared laser beam passes through the gas cell, the gas cell configured to receive the gas sample; a gas chromatography column assembly including an input port, a gas chromatography column, and an output port, the gas chromatography column assembly configured to provide the gas sample to the Fabry-Perot gas cell; and a controller in electronic communication with the infrared sensor and configured to process the received infrared laser beam to estimate a molecular composition or an isotopic ratio of the gas sample.

A second embodiment may include the first embodiment, wherein the Fabry-Perot gas cell comprises a macro Fabry-Perot gas cell having a cavity volume in a range from 0.1 to about 5 milliliters and a cavity length in a range from about 0.5 to about 10 cm.

A third embodiment may include the first embodiment, wherein the Fabry-Perot gas cell comprises a micro Fabry-Perot gas cell having a cavity volume less than about 5 microliters and a cavity length less than about 2 mm.

A fourth embodiment may include any one of the first through third embodiments, further comprising a first optical fiber coupling the infrared laser and the Fabry-Perot gas cell; and a second optical fiber coupling the Fabry-Perot gas cell and the infrared sensor.

A fifth embodiment may include the fourth embodiment, wherein a first mirror is integrated into an end of the first optical fiber in the Fabry-Perot gas cell and a second mirror is integrated into an end of the second optical fiber in the Fabry-Perot gas cell.

A sixth embodiment may include any one of the first through fifth embodiments, wherein the Fabry-Perot gas cell has a cavity length that is an integer multiple of a difference between first and second peaks in an isotopic measurement.

A seventh embodiment may include any one of the first through sixth embodiments, wherein the infrared laser is a tunable laser and is configured to emit an infrared laser beam having a wavelength in a range from 1 to 2 μm.

An eighth embodiment may include any one of the first through seventh embodiments, wherein the infrared laser comprises a plurality of tunable infrared lasers; and each of the plurality of tunable infrared lasers is configured to emit a corresponding infrared laser beam having a preselected wavelength corresponding to an absorption peak of a distinct chemical compound in the gas sample.

A ninth embodiment may include the eighth embodiment, wherein the infrared sensor comprises a plurality of infrared sensors corresponding to the plurality of tunable infrared lasers.

A tenth embodiment may include any one of the first through ninth embodiments, wherein the infrared laser, the infrared sensor, and the Fabry-Perot gas cell are collectively configured to detect methane or carbon dioxide isotopes in the gas sample.

An eleventh embodiment may include any one of the first through tenth embodiments, wherein the infrared laser, the infrared sensor and the Fabry-Perot gas cell are collectively configured to detect permanent gases in the gas sample.

A twelfth embodiment may include any one of the first through eleventh embodiments, wherein the gas chromatography column assembly further comprises a gas chromatography detector in electronic communication with the controller, the gas chromatography detector being selected from a flame ionization detector, a thermal conductivity detector, and an electron capture detector.

A thirteenth embodiment may include any one of the first through twelfth embodiments, wherein the input port comprises an auto-injector with a sample loop having a volume of less than 5 ml.

A fourteenth embodiment may include any one of the first through thirteenth embodiments, wherein the gas chromatography column comprises a capillary column including thin films of a stationary phase coating the inner surface of the gas chromatography column.

A fifteenth embodiment may include any one of the first through fourteenth embodiments, further comprising a pressure controller configured to regulate a pressure of the gas sample in the Fabry-Perot gas cell such that the pressure of the gas sample is less than 100 mbar.

In a sixteenth embodiment a method for evaluating a composition of an oilfield gas sample, the method comprising auto-injecting the gas sample into a gas chromatography assembly including a gas chromatography column; receiving the gas sample in a Fabry-Perot gas cell from the gas chromatography column; emitting infrared radiation into the Fabry-Perot gas cell using an infrared laser; receiving the infrared radiation from the Fabry-Perot gas cell at an infrared sensor; and estimating a composition of the gas sample from the infrared radiation received at the infrared sensor.

A seventeenth embodiment may include the sixteenth embodiment, wherein the estimating a composition rises estimating an isotopic ratio of methane in the gas sample.

An eighteenth embodiment may include the seventeenth embodiment, wherein the Fabry-Perot gas cell has a cavity length that is an integer multiple of a difference between first peak of carbon-13 methane and a second peak of carbon-12 methane.

A nineteenth embodiment may include any one of the sixteenth through eighteenth embodiments, wherein the emitting infrared radiation comprises emitting infrared radiation having a wavelength in a range from 1 to 2 μm.

A twentieth embodiment may include any one of the sixteenth through nineteenth embodiments, wherein the emitting infrared radiation comprises emitting first infrared radiation having a first wavelength using a first infrared laser and emitting second infrared radiation having a second wavelength using a second infrared laser; and the method further comprises using an optical switch to modulate the infrared radiation received by the Fabry-Perot gas cell between the first infrared radiation and of the second infrared radiation.

Although an apparatus for fast gas chromatography and infrared spectroscopy measurements of oilfield fluids has been described in detail, it should be understood that various changes, substitutions and alternations can be made herein without departing from the spirit and scope of the disclosure as defined by the appended claims. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The terms “approximately,” “about,” and “substantially” as used herein represent an amount close to the stated amount that is within standard manufacturing or process tolerances, or which still performs a desired function or achieves a desired result. For example, the terms “approximately,” “about,” and “substantially” may refer to an amount that is within less than 5% of, within less than 1% of, within less than 0.1% of, and within less than 0.01% of a stated amount.

1. An apparatus for making fast gas chromatography measurements of an oilfield gas sample, the apparatus comprising:
an infrared laser configured to emit an infrared laser beam;
an infrared sensor configured to receive the infrared laser beam;
a Fabry-Perot gas cell deployed in a path between the infrared laser and the infrared sensor such that the infrared laser beam passes through the gas cell, the gas cell configured to receive the gas sample;
a gas chromatography column assembly including an input port, a gas chromatography column, and an output port, the gas chromatography column assembly configured to provide the gas sample to the Fabry-Perot gas cell; and
a controller in electronic communication with the infrared sensor and configured to process the received infrared laser beam to estimate a molecular composition or an isotopic ratio of the gas sample.

2. The apparatus of claim 1, wherein the Fabry-Perot gas cell comprises a macro Fabry-Perot gas cell having a cavity volume in a range from 0.1 to about 5 milliliters and a cavity length in a range from about 0.5 to about 10 cm.

3. The apparatus of claim 1, wherein the Fabry-Perot gas cell comprises a micro Fabry-Perot gas cell having a cavity volume less than about 5 microliters and a cavity length less than about 2 mm.

4. The apparatus of claim 1, further comprising:
a first optical fiber coupling the infrared laser and the Fabry-Perot gas cell; and
a second optical fiber coupling the Fabry-Perot gas cell and the infrared sensor.

5. The apparatus of claim 4, wherein a first mirror is integrated into an end of the first optical fiber in the Fabry-Perot gas cell and a second mirror is integrated into an end of the second optical fiber in the Fabry-Perot gas cell.

6. The apparatus of claim 1, wherein the Fabry-Perot gas cell has a cavity length that is an integer multiple of a difference between first and second peaks in an isotopic measurement.

7. The apparatus of claim 1, wherein the infrared laser is a tunable laser and is configured to emit an infrared laser beam having a wavelength in a range from 1 to 2 μm.

8. The apparatus of claim 1, wherein:
the infrared laser comprises a plurality of tunable infrared lasers; and
each of the plurality of tunable infrared lasers is configured to emit a corresponding infrared laser beam having a preselected wavelength corresponding to an absorption peak of a distinct chemical compound in the gas sample.

9. The apparatus of claim 8, wherein the infrared sensor comprises a plurality of infrared sensors corresponding to the plurality of tunable infrared lasers.

10. The apparatus of claim 1, wherein the infrared laser, the infrared sensor, and the Fabry-Perot gas cell are collectively configured to detect methane or carbon dioxide isotopes in the gas sample.

11. The apparatus of claim 1, wherein the infrared laser, the infrared sensor and the Fabry-Perot gas cell are collectively configured to detect permanent gases in the gas sample.

12. The apparatus of claim 1, wherein the gas chromatography column assembly further comprises a gas chromatography detector in electronic communication with the controller, the gas chromatography detector being selected from a flame ionization detector, a thermal conductivity detector, and an electron capture detector.

13. The apparatus of claim 1, wherein the input port comprises an auto-injector with a sample loop having a volume of less than 5 ml.

14. The apparatus of claim 1, wherein the gas chromatography column comprises a capillary column including thin films of a stationary phase coating the inner surface of the gas chromatography column.

15. The apparatus of claim 1, further comprising a pressure controller configured to regulate a pressure of the gas sample in the Fabry-Perot gas cell such that the pressure of the gas sample is less than 100 mbar.

16. A method for evaluating a composition of an oilfield gas sample, the method comprising:
auto-injecting the gas sample into a gas chromatography assembly including a gas chromatography column;
receiving the gas sample in a Fabry-Perot gas cell from the gas chromatography column;
emitting infrared radiation into the Fabry-Perot gas cell using an infrared laser;
receiving the infrared radiation from the Fabry-Perot gas cell at an infrared sensor; and
estimating a composition of the gas sample from the infrared radiation received at the infrared sensor.

17. The method of claim 16, wherein the estimating a composition rises estimating an isotopic ratio of methane in the gas sample.

18. The method of claim 17, wherein the Fabry-Perot gas cell has a cavity length that is an integer multiple of a difference between first peak of carbon-13 methane and a second peak of carbon-12 methane.

19. The method of claim 16, wherein the emitting infrared radiation comprises emitting infrared radiation having a wavelength in a range from 1 to 2 μm.

20. The method of claim 16, wherein:
the emitting infrared radiation comprises emitting first infrared radiation having a first wavelength using a first infrared laser and emitting second infrared radiation having a second wavelength using a second infrared laser; and
the method further comprises using an optical switch to modulate the infrared radiation received by the Fabry-Perot gas cell between the first infrared radiation and of the second infrared radiation.
